(19) United States
(12) Patent Application Publication
(10) Pub. No.: US 2025/0099393 A1
(43) Pub. Date: Mar. 27, 2025

(54) IONIZABLE LIPIDS, LIPID NANOPARTICLES, AND USES THEREOF

(71) Applicant: Verily Life Sciences LLC, South San Francisco, CA (US)

(72) Inventors: Zhan Wang, San Jose, CA (US); Nicole Peck, Redwood City, CA (US); Sarah Sanowar, San Francisco, CA (US); Manali Dwarakanath, Fremont, CA (US)

(21) Appl. No.: 18/832,440

(22) PCT Filed: Jan. 23, 2023
(86) PCT No.: PCT/US2023/061089

§ 371 Date: Jul. 23, 2024

Related U.S. Application Data
(60) Provisional application No. 63/267,084, filed on Jan. 24, 2022.

(51) Int. Cl. A61K 9/51; A61K 9/1271; A61K 48/00; C12N 9/22; C12N 15/11; C12N 15/88

(52) U.S. Cl. CPC A61K 9/5123; A61K 9/1271; A61K 48/0041; C12N 9/22; C12N 15/111; C12N 15/88; C12N 2310/20; C12N 2310/321; C12N 2310/322

(57) ABSTRACT

Ionizable lipids and lipid nanoparticles comprising ionizable lipids are provided herein. Also provided are methods for making and using the lipid nanoparticles for delivery to a cell one or more payload agents, including polypeptides, mRNAs such as mRNA encoding Cas9 nuclease, and guide RNAs comprising backbone, sugar or base modifications.

### PRIOR RELATED APPLICATION

This application claims the benefit of and priority to U.S. Provisional Application No. 63/267,084, filed on Jan. 24, 2022, which is hereby incorporated by reference in its entirety.

### BACKGROUND

Gene therapy and vaccination rely on the effective, specific, and tolerable delivery of therapeutic agents in vivo. Lipid nanoparticles (LNPs) for in vivo delivery have emerged as promising delivery vehicles for numerous therapeutic applications. However, LNPs with improved therapeutic efficacy and minimal toxicity for applications such as gene editing are still needed.

### SUMMARY

Provided herein are ionizable lipids and LNPs that comprise the ionizable lipids for in vitro, ex vivo or in vivo delivery of an agent. For example, described herein is a lipid nanoparticle (LNP) comprising an ionizable lipid of Formula I,

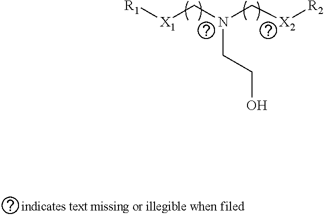

wherein, R₁ and R₂ are independently selected from the group consisting of:

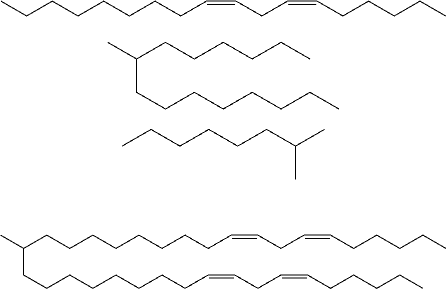

wherein X₁ and X₂ are hydrolyzable linkers, wherein (a) is 2-9 (i.e., 2, 3, 4, 5, 6, 7, 8, or 9 carbons), and wherein (b) is 2-9 (i.e., 2, 3, 4, 5, 6, 7, 8, or 9 carbons). In some embodiments, the hydrolyzable linker in the LNP is an ester. In some embodiments, the LNP comprises the ionizable lipid of Formula II,

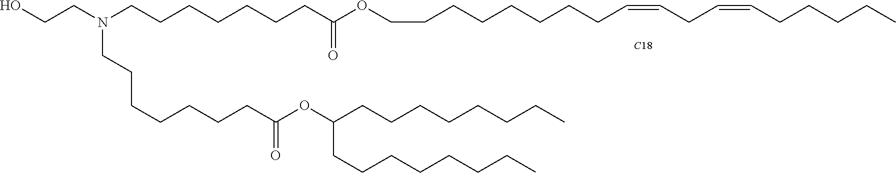

In some embodiments, the LNP further comprises cholesterol, a helper phospholipid and a polyethylene glycol (PEG)-lipid. In some embodiments, about 20% to about 80% of the total lipid content of the LNP is the ionizable lipid of Formula I or Formula II.

In some embodiments, the LNP comprises (a) 20%-80% of the ionizable lipid of Formula I; (b) 5%-20% of a helper lipid, (c) 10%-60% cholesterol; and (d) 0.5%-5% polyethylene glycol (PEG).

In some embodiments, the LNP further comprises a polypeptide, a nucleic acid, or a combination thereof. In some embodiments, a nucleic acid encoding a heterologous polypeptide is encapsulated in the LNP. In some embodiments, the nucleic acid is an mRNA. In some embodiments, the mRNA encodes an RNA-guided endonuclease. In some embodiments, the RNA-guided endonuclease is a Cas9 nuclease. In some embodiments, the LNP comprising an mRNA encoding an RNA-guided endonuclease further comprises a guide RNA (gRNA). The guide RNA can be a modified guide RNA. Optionally, the guide RNA comprises one or more modifications selected from the group consisting of a backbone modification, a sugar modification, and a base modification. By way of example, the sugar modification can be a 2′ OMe or a 2′F modification. In some embodiments, the backbone modification is a thioester modification. Optionally, the gRNA comprises a nucleic acid sequence of 100 nucleotides, wherein 69 of the 100 nucleotides are modified.

The mRNA encoding the RNA-guided endonuclease and the guide RNA can be encapsulated separately and then combined to form the LNP. In some embodiments, the mRNA encoding the RNA-guided endonuclease and the guide RNA are co-encapsulated in the LNP. Optionally, the ratio of nitrogen groups from the ionizable lipid to phosphate groups in the mRNA and/or gRNA in the LNP is from about 4 to about 6. In some embodiments, the LNP further comprises at least one nucleic acid template.

Also provided is a plurality of any of the LNPs described herein. Further provided is a pharmaceutical composition comprising one or more of the LNPs described herein.

Further provided is a method of delivering a heterologous polypeptide to a cell comprising contacting the cell with one or more LNPs comprising a heterologous polypeptide, or contacting a cell with one or LNPs comprising a nucleic acid encoding a heterologous polypeptide described herein. Also provided is a method of delivering a heterologous polypeptide to a cell comprising contacting the cell with a pharmaceutical composition comprising one or more LNPs comprising a heterologous polypeptide, or contacting the cell with a pharmaceutical composition comprising one or more LNPs comprising a nucleic acid encoding a heterologous polypeptide described herein.

Also provided is a method of editing the genome of a cell comprising contacting the cell with one or more LNPs comprising a nucleic acid, wherein the nucleic acid encodes an RNA-guided endonuclease and a guide RNA described herein, or contacting the cell with one ore more LNPs comprising a ribonucleoprotein complex (RNP), wherein the RNP comprises an RNA-guided endonuclease and a guide RNA as described herein. Further provided is a method of editing the genome of a cell by contacting a cell with a pharmaceutical composition, wherein the pharmaceutical composition comprises one or more LNPs comprising a nucleic acid encoding an RNA-guided endonuclease and a guide RNA as described herein, or contacting the cell with a pharmaceutical composition comprising one or more LNPs comprising a ribonucleoprotein complex (RNP), the RNP including an RNA-guided endonuclease and a guide RNA as described herein. The cell is optionally edited in vitro, ex vivo or in vivo. In some embodiments, the cell is a human cell.

Also provided are cells comprising one or more LNPs described herein, as well as genetically modified cells made by any of the methods described herein.

### DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows a comparison between Formulation 1 (F1) (a formulation comprising ionizable lipid PCTA-1 (Formula II)), Comparative Formulation 1 (CF1), and Comparative Formulation 2 (CF2). All formulations packaged Cas9 mRNA and an sgRNA targeting either TTR or PCSK9. When targeting TTR (first three columns from the left), F1 (the formulation comprising PCTA-1) achieved the highest editing, showing improved editing over CF2 and a large (about 5 times higher) improvement over CF1. When targeting PCSK9 (first three columns from the right) F1 achieved significantly higher editing (2-3 times higher) than both CF2 and CF1 when using the same sgRNA.

FIG. 2 shows TTR sgRNA sequences (Guide 1 (SEQ ID NO: 1), Guide 2 (SEQ ID NO: 1), Guide 3 (SEQ ID NO. 2), and Guide 4 (SEQ ID NO. 3)) with selected chemical modifications. Capitalized bases are ribonucleotides. Lower case bases are 2′-OMe ribonucleotides. Guide 2 includes 2′ deoxy-2′-fluoro-ribonucleotides, as underlined in SEQ ID NO: 1 (ttacagccac gtctacagca gttttagagc tagaaatage aagttaaaat aaggctagtc cgttatcaac ttgaaaaagt ggcaccgagt cggtgctttt).

FIG. 3A-B provide the results of an in vitro primary mouse hepatocyte cell screening showing (FIG. 3A) similar editing for all guide RNAs set forth in FIG. 2, and (FIG. 3B) a reduction in cell supernatant protein levels for all guide RNAs set forth in FIG. 2.

FIG. 4A-B provide in vivo results for a literature nanoparticle formulation (FIG. 4A) and a comparison of literature formulations against a nanoparticle formulation comprising PCTA-1 (Formula II) (FIG. 4B).

### DETAILED DESCRIPTION

Provided herein are ionizable lipids and lipid nanoparticles (LNPs) comprising ionizable lipids that can be used for delivery of an agent to one or more cells, for example, for delivery of a nucleic acid, a polypeptide, or a combination thereof, to a cell. As used herein, an ionizable lipid is a lipid molecule that is neutral at physiological pH, but is protonated at low pH, to make it positively charged. The ionizable lipids described herein employ a combination of branched alkyl and linoleyl tails, with a pKa-tuned ethanolamine headgroup, to enable efficient packing, delivery, and release of the agent from LNPs. The ionizable lipids also comprise hydrolyzable linkers to limit toxicity by allowing for rapid lipid clearance after LNP disruption, for example, after delivery of the LNP(s) to a cell.

Provided herein is an ionizable lipid having Formula I,

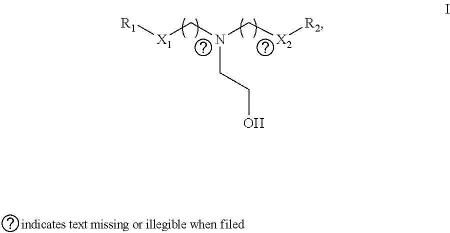

wherein, R₁ and R₂ are independently selected from the group consisting of

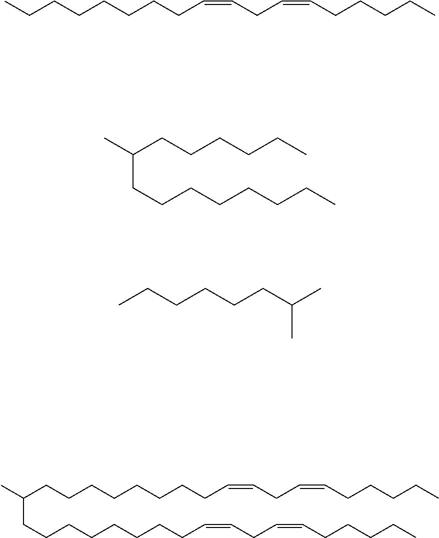

wherein X₁ and X₂ are hydrolyzable linkers, wherein (a) is 2-9, and wherein (b) is 2-9. In some embodiments, the hydrolyzable linker is an ester. In some embodiments, the ionizable is a lipid of Formula II (PCTA-1),

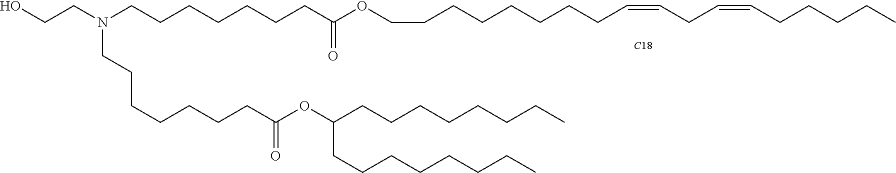

Also provided are LNPs comprising any of the ionizable lipids described herein. For example, provided herein is an LNP comprising the ionizable lipid of Formula I or Formula II. The ionizable lipid of Formula I or Formula II has a unique combination of chemical features that allows successful delivery of payloads (e.g., nucleic acids, polypeptides, small molecules, etc.). The ethanolamine headgroup gives a pKa in the 6.2-6.8 range, such that the amine is uncharged at physiological pH, but positively charged at acidic pH. Further, the ester linkages are hydrolyzable, and thus enable rapid clearance of the lipid after delivery of the payload. The unsaturation in the linoleyl tail disrupts orderly membrane packing, enabling the LNP to release the payload and escape the endosome more readily. The branched alkyl tail serves a similar function. The combination of these two tails with the tuned pKa of the ethanolamine headgroup enables efficient payload packing, delivery, and release, while the hydrolyzable linkers limit toxicity by allowing for rapid lipid clearance after LNP disruption.

As used herein, lipid nanoparticles (LNPs) are spherical vesicles in the nanometer range. Generally, lipid nanoparticles are taken up by cells via endocytosis, and the ionizability of the lipids at low pH enables endosomal escape, which allows release of the cargo into the cytoplasm. In addition, lipid nanoparticles optionally contain a helper lipid to promote cell binding, cholesterol to fill the gaps between the lipids, and/or a polyethylene glycol (PEG) to reduce opsonization by serum proteins and reticuloendothelial clearance. LNPs comprising ionizable lipids are capable of carrying nucleic acids, polypeptides, drugs or other substances, which are either encapsulated by or contained within the lipid layer. LNPs can vary in size, i.e., diameter. For example, an LNP can have a size of about 1000 nanometers (nm) or less. For example, an LNP can have a size of about 50 nm to about 1000 nm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, or about 50 nm to about 100 nm. The LNPs described herein include LNPs comprising ionizable lipids and one or more payload agents, for example, a nucleic acid (e.g., a mRNA, a gRNA, a siRNA, an antisense molecule, or an aptamer), a polypeptide, a ribnonucleoprotein complex (e.g., an RNA-guided endonuclease complexed with a gRNA), a drug, or a small molecule, to name a few. Combinations of these agents, for example, an mRNA and a guide RNA, can also be within any of the LNPs described herein. As used throughout, an encapsulated agent is an agent that is completely or partially located in the interior aqueous or non-aqueous space of the LNP. For example, in the LNPs described herein, at least about 75%, 80%, 85%, 90%, 95% or 99% of the agent is incorporated into the interior space of the LNP.

Pluralities or populations of two or more of the LNPs described herein are also provided. For example, a plurality of LNPs can comprise from about two to about 1×10¹⁴ (100 trillion) LNPs. For example, a plurality can have at least 100, 250, 500, 750, 1000, 5000, 10,000, 25,000, 50,000, 100,000, 500,000, 1 million or more LNPs. The LNPs in the plurality optionally differ in payloads, type of ionizable lipid, and/or size. LNPs can be made by any suitable method known to or later discovered by one of skill in the art.

Exemplary methods for the preparation of LNPs are described in Parhi and Suresh (“Preparation and characterization of solid lipid nanoparticles—a review,” Curr. Drug Discov. Technol. 9(1): 2-16 (2012)) and Corrias and Lai (“New methods for lipid nanoparticles preparation,” Recent. Pat. Drug. Deliv. Formul. 5(3): 201-213 (2011)), which are hereby incorporated by reference in their entireties.

In some embodiments, about 20% to about 80% of the total lipid content of any of the LNPs described herein is an ionizable lipid, e.g., the ionizable lipid of Formula I or the ionizable lipid of Formula II. Optionally, about 20%, 25%, 30%, 35%, 40%, 45%, 50%, 55%, 60%, 65%, 70%, 75%, 80% or any percentage in between these percentages of the total lipid content of the LNP can be the ionizable lipid of Formula I or the ionizable lipid of Formula II. Any of the LNPs comprising the ionizable lipid of Formula I or Formula II can further comprise one or more additional lipids. In general, a variety of lipid components can be used. These include neutral lipids that exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. Synthetic derivatives of any of the lipids described herein can also be used to make lipid nanoparticles. Lipid nanoparticles can also comprise a sterol, for example, cholesterol. Lipid nanoparticles can also comprise a cationic lipid that carries a net positive charge at about physiological pH. Such cationic lipids include, but are not limited to, N,N-dioleyl-N,N-dimethylammonium chloride (DODAC); N-(2,3-dioleyloxy) propyl-N,N—N-triethylammonium chloride (DOTMA); N,N-distearyl-N,N-dimethylammonium bromide (DDAB); N-(2,3-dioleoyloxy) propyl)-N,N,N-trimethylammonium chloride (DOTAP); 1,2-Dioleyloxy-3-trimethylaminopropane chloride salt (DOTAP·Cl); 3.beta.-(N—(N′,N′-dimethylaminoethane)-carbamoyl) cholesterol (“DC-Chol”), N-(1-(2,3-dioleyloxy) propyl)-N-2-(sperminecarboxamido)ethyl)-N,N-dimethyl-ammonium trifluoracetate (“DOSPA”), dioctadecylamidoglycyl carboxyspermine (DOGS), 1,2-dileoyl-sn-3-phosphoethanolamine (DOPE), 1,2-dioleoyl-3-dimethylammonium propane (DODAP), N,N-dimethyl-2,3-dioleyloxy) propylamine (DODMA), and N-(1,2-dimyristyloxyprop-3-yl)-N,N-dimethyl-N-hydroxyethyl ammonium bromide (DMRIE). Anionic lipids are also suitable for use in lipid nanoparticles described herein. These include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

In some examples, the LNP comprises phosphatidylcholine, phosphatidylethanolamine, phosphatidylserine, phosphatidylinositol, phosphatidic acid, phosphatidylglycerol, palmitoyloleoyl phosphatidylcholine, lysophosphatidylcholine, lysophosphatidylethanolamine, dimyristoylphosphatidylcholine (DMPC), dipalmitoylphosphatidylcholine (DPPC), dioleoylphosphatidylcholine, distearoylphosphatidylcholine (DSPC), dilinoleoylphosphatidylcholine, a 1,2-distearoyl-sn-glycero-3-phosphoethanolamine (DSPE) conjugated polyethylene glycol (DSPE-PEG), a sphingomyelin, cholesterol, or any combination thereof. In some embodiments, PEG can be PEG-molecular weight (MW500) to PEG-MW20000.

In some embodiments, the LNP comprises an ionizable lipid having Formula I or Formula II, cholesterol, a helper phospholipid and a polyethylene glycol lipid, for example, pegylated myristoyl diglyceride (DMG-PEG2000). In some embodiments, the LNP comprises an ionizable lipid having Formula I or Formula II, cholesterol, DSPC, and DMG-PEG2000. Optionally, the LNP comprises about 45% to 55% ionizable lipid having Formula I or Formula II, about 35% to about 45% cholesterol, about 5% to about 10% DSPC, and about 1% to about 5% DMG-PEG2000. In some embodiments, the LNP comprises about 50% ionizable lipid having Formula I or Formula II, about 38% cholesterol, about 10% DSPC, and about 2% DMG-PEG2000.

Optionally, the LNP comprises about 20%, 25%, 30%, 35%, 40%, 45%, 50%, 55%, 60%, 65%, 70%, 75%, 80% or any percentage in between these percentages of an ionizable lipid of Formula I or Formula II; about 5%, 10%, 15%, 20% or any percentage in between these percentages of a helper lipid; about 10%, 15%, 20%, 25%, 30%, 35%, 40%, 45%, 50%, 55%, 60%, 65%, 70%, 75%, 80%, or any percentage in between these percentages of cholesterol; and about 0.5%, 1%, 1.5%, 2.0%, 2.5%, 3.0%, 3.5%, 4.0%, 4.5%, 5.0%, 5.5%, or any percentage in between these percentages of PEG.

The LNP optionally comprises a targeting molecule or targeting moiety attached to or incorporated within the lipid layer of the LNP. The targeting molecule or moiety can be used to target the LNPs to a particular cell or tissue in vitro, ex vivo, or in vivo, for cellular delivery of an payload agent encapsulated in the LNP. As used throughout, a targeting molecule is a molecule that has a binding affinity for an antigen on a cell, optionally a specific binding affinity, and can include, but is not limited to, an antibody, a polypeptide, a peptide, an aptamer or a small molecule. As used herein, an antigen on a cell can be, but is not limited to a protein, a lipid or a carbohydrate.

### LNPs Comprising Agents

The LNPs described herein include LNPs comprising ionizable lipids and a payload agent. The agent can be, for example, a nucleic acid (e.g., a mRNA, a gRNA, a siRNA, an antisense molecule), a polypeptide, a ribnonucleoprotein complex (e.g., an RNA-guided endonuclease complexed with a guide RNA), a drug, or a small molecule that is encapsulated (partially or fully) in the LNP. In some embodiments, the agent is a polypeptide. As used throughout, polypeptide, protein and peptide are used interchangeably to refer to a polymer of amino acid residues. As used herein, the terms encompass amino acid chains of any length, including full-length proteins, wherein the amino acid residues are linked by covalent peptide bonds.

In some embodiments, the encapsulated agent is a nucleic acid. As used throughout, the term nucleic acid refers to deoxyribonucleic acids (DNA) or ribonucleic acids (RNA) and polymers thereof in either single- or double-stranded form. In some embodiments, the nucleic acid comprises an exon sequence, an intron sequence, a regulatory sequence (for example, a promoter, enhancer, or a silencer), a transcriptional control sequence, a translational control sequence, a splicing site, or a non-coding sequence. Optionally, the nucleic acid is a nucleic acid encoding a polypeptide, for example, a DNA molecule encoding a polypeptide or an RNA molecule (e.g., mRNA) encoding a polypeptide. Optionally, the nucleic acid encoding a polypeptide can be operably linked to one or more of an intron sequence, a regulatory sequence (for example, a promoter, enhancer, or a silencer), a transcriptional control sequence, a translational control sequence, a splicing site, or a non-coding sequence. As used herein, a nucleic acid is operably linked when it is placed into a functional relationship with another nucleic acid sequence. For example, a promoter or enhancer is operably linked to a coding sequence if it affects the transcription of the coding sequence and a ribosome binding site is operably linked to a coding sequence if it is positioned so as to facilitate translation of the coding sequence.

Optionally, the mRNA encodes an RNA-guided endonuclease. The CRISPR/Cas system, an RNA-guided nuclease system that employs a Cas endonuclease, can be used to edit the genome of a host cell or organism. The CRISPR/Cas system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

As used herein, the term Cas9 or Cas9 nuclease refers to an RNA-mediated nuclease (e.g., of bacterial or archeal origin or derived from a bacterial or archeal nuclease). Exemplary RNA-mediated nucleases include the foregoing Cas9 proteins and homologs thereof. Other RNA-mediated nucleases include Cpf1 (see, e.g., Zetsche et al. (2015) Cell 163(3): 759-771), Cas13-based RNA editors, and homologs thereof.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chloroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi et al. (2013) RNA Biol. 10(5): 726-737; Makarova et al. (2011) Nat. Rev. Microbiol. 9(6): 467-477; Hou et al. (2013) Proc Natl Acad Sci USA 110(39): 15644-49; Sampson et al. (2013) Nature 497(7448): 254-7; and Jinek et al. (2012) Science 337(6096): 816-21. Variants of any of the Cas9 nucleases provided herein can be optimized for efficient activity or enhanced stability in the host cell. Thus, engineered Cas9 nucleases are also contemplated. See, for example, Slaymaker et al. (2016) Rationally engineered Cas9 nucleases with improved specificity, Science 351 (6268): 84-88.

The Cas9 endonuclease optionally binds a target nucleic acid to create a double-stranded break. However, the Cas9 protein can be a nickase, such that when bound to ta arget nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation. See, for example, Ran et al. (2013), Double nicking by RNA-guided CRISPR Cas9 for enhanced genome editing specificity, Cell 154 (6): 1380-1389.

Also provided herein are LNPs comprising one or more guide RNAs or one or more nucleic acids encoding a guide RNA. As used throughout, a guide RNA (gRNA) sequence is a sequence that interacts with an RNA-guided nuclease and specifically binds to or hybridizes to a target nucleic acid within the genome of a cell, such that the gRNA and the targeted nuclease co-localize to the target nucleic acid in the genome of the cell. The degree of complementarity between a guide RNA and a genomic sequence can be about 70%, 75%, 80%, 85%, 90%, 95%, 97%, 98%, 99%, or 100%.

In some instances, the guide RNA can be operably linked to at least one transcriptional or regulatory control sequence, for example, a promoter. Each gRNA includes a DNA targeting sequence or protospacer sequence of about 10 to 50 nucleotides in length that specifically binds to or hybridizes to a target DNA sequence in the genome. For example, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. Optionally, the gRNA does not comprise a tracrRNA sequence.

In some embodiments, the LNPs comprise an mRNA encoding an RNA-guided endonuclease and a guide RNA. The mRNA encoding the RNA-guided endonuclease and the guide RNA can be encapsulated in separate LNPs that are then combined to form a mixed population of LNPs comprising either the mRNA encoding the RNA-guided endonuclease or the guide RNA in different LNPs. In some embodiments, the mRNA encoding the RNA-guided endonuclease and the guide RNA are co-encapsulated in the same LNPs. Optionally, the ratio of nitrogen groups of the ionizable lipid of the LNP to phosphate groups in the mRNA and/or gRNA in the LNP is from about 4 to about 6.

The guide RNA is optionally a modified guide RNA. The one or more modifications to the guide RNA can be selected from the group consisting of a backbone modification, a sugar modification, and a base modification. Optionally, the guide RNA has one or more sugar modifications, one or more base modifications, and/or one or more backbone modifications.

In some guide RNAs, the sugar modification is a modification of the 2′ position of the ribose group. In some guide RNAs, the modification of the 2′ position is selected from 2′-O-methyl (2′-OMe) which replaces the 2′ hydroxyl group with a methyl group, and 2′-fluoro (2′-F). which replaces the 2′ hydroxyl group with a fluoride.

In some guide RNAs, the phosphate backbone modification is a thioester modification (e.g., a phosphorothioate linkage). In some instances, at least two, three, four, five, six, seven, eight, nine, ten, or more of the nucleotides in the guide RNA are modified nucleotides. In certain instances, at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 of the nucleotides in the guide RNA sequence are modified. In some embodiments, the guide RNA comprises a nucleic acid sequence of 100 nucleotides, wherein 69 of the 100 nucleotides are modified using the modification pattern shown below:

Optionally, the LNP comprises a ribonucleotide protein complex (RNP), wherein the RNP comprises an RNA-guided nuclease (e.g., Cas9 nuclease) and a guide RNA. In other embodiments, the LNP comprises an RNP, wherein the RNP comprises an RNA-guided nuclease (e.g., Cas9 nuclease), a guide RNA, and one or more template nucleic acids for repair or recombination in the genome of a cell. The template can be used to alter or insert a nucleic acid sequence at or near a target site for the RNA-guided endonuclease. In some embodiments, the template is a heterologous nucleic acid sequence encoding a polypeptide, an exon sequence, an intron sequence, a regulatory sequence, a transcriptional control sequence, a translational control sequence, a splicing site, or a non-coding sequence, to name a few. Optionally, the template is a single stranded, double-stranded, or partially double stranded template. In some embodiments, the template can be used to repair a genomic sequence via homology-directed repair, or via non-homologous end joining.

As used herein, the term heterologous refers to what is not normally found in nature. The term heterologous nucleic acid sequence refers to a nucleic acid sequence not normally found in a given cell in nature. As such, a heterologous nucleic acid sequence may be (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) naturally found in the host cell but positioned outside of its natural locus.

In the LNPs described herein, the template can be 10, 15, 20, 25, 50, 75, 100, 150, 200, 500, 1000, 1500, 2000, 2500, 3000, 3500, 4000, 4500, 5000, 5500, 6000, or more nucleotides in length. In some embodiments, the template can be flanked by homology arms, i.e., nucleic acid sequences that are complementary to sequences located upstream and downstream of an RNA-guided endonuclease cleavage site in the genome of a cell. As used herein, the term complementary or complementarity refers to specific base pairing between nucleotides or nucleic acids. Complementary nucleotides are, generally, A and T (or A and U), and G and C. In some embodiments, the degree of complementarity between a nucleic acid sequence (for example, a guide RNA or a homology arm) and a genomic sequence can be about 70%, 75%, 80%, 85%, 90%, 95%, 97%, 98%, 99%, or 100%.

Also provided herein are one or more cells comprising any of the LNPs or a plurality of LNPs described herein. Further provided are compositions comprising one or more of the LNPs described herein. Pharmaceutical compositions include, for example, a therapeutically effective amount of any of the LNPs described herein and a pharmaceutical carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Optionally, the composition comprising one or more LNPs is in a kit, which includes for example, a container for the composition or components thereof, a device for administering the composition, and/or a mixer or mixing device for making the composition.

Pharmaceutical compositions comprising any of the LNPs described herein can be prepared according to standard techniques and can further comprise a pharmaceutically acceptable carrier. Generally, normal saline is employed as the pharmaceutically acceptable carrier. Other suitable carriers include, e.g., water, buffered water or saline, 0.4% saline, 0.3% glycine, dextrose, and the like, including glycoproteins for enhanced stability, such as albumin, lipoprotein, and globulin. These compositions are usually sterile. The pharmaceutical compositions can also contain a pharmaceutically acceptable excipient. Such excipients include any pharmaceutical agent that does not itself induce an immune response harmful to the individual receiving the composition, and which may be administered without undue toxicity. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. The preparation of pharmaceutically acceptable carriers, excipients and formulations containing these materials is described in, e.g., Remington: The Science and Practice of Pharmacy, 22nd edition, Loyd V. Allen et al, editors, Pharmaceutical Press (2012).

Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the LNP suspension can include lipid-protective agents which protect lipids against free-radical and lipid-peroxidative damages on storage. Lipophilic free-radical quenchers, such as alphatocopherol and water-soluble iron-specific chelators, such as ferrioxamine, are suitable.

The concentration of the LNPs in the pharmaceutical formulations can vary widely, i.e., from less than about 0.05% to about 2-5% or 10 to 30% by weight. The concentration can be selected primarily by fluid volumes, viscosities, in accordance with the particular mode of administration selected. The LNPs can also be dried or lyophilized and resuspended to a desired concentration in water or buffers at time of use.

### Methods

Provided herein are methods for delivering a polypeptide, for example, a recombinant polypeptide, to a cell. The methods comprise contacting a cell with one or more of the LNPs comprising a heterologous polypeptide described herein or contacting the cell with one or more of the LNPs comprising a nucleic acid encoding a heterologous polypeptide described herein. Optionally, the cell can be contacted with a pharmaceutical composition comprising the LNP.

Also provided is a method of editing the genome of a cell comprising contacting the cell with an LNP comprising a nucleic acid encoding an RNA-guided nuclease and a guide RNA, wherein the guide RNA interacts with the RNA-guided nuclease such that the RNA-guided nuclease specifically binds and cleaves a target DNA in the genome. As used throughout, the term editing, in the context of editing a genome of a cell, refers to inducing a structural change in the sequence of the genome at a target genomic region. For example, the editing or modifying can take the form of inserting a nucleotide sequence (e.g., a nucleic acid template) into the genome of the cell. Such editing can be performed, for example, by inducing a double stranded break within a target genomic region, or a pair of single stranded nicks on opposite strands and flanking the target genomic region. Methods for inducing single or double stranded breaks at or within a target genomic region include the use of an RNA-guided endonuclease, or a derivative thereof, and a guide RNA, or pair of guide RNAs, directed to the target genomic region, as described herein. Any of the editing methods described herein can be used to achieve at least about 10%, 20%, 30%, 40%, 50%, 60%, 70%, or 80% editing efficiency in a population of cells. Also provided are genetically modified cells produced by any of the methods described herein.

In any of the delivery methods described herein, the agent(s) encapsulated by the LNPs is introduced into the cell, after contacting the cell with the LNP. As used herein, introducing in the context of introducing a nucleic acid, a polypeptide, or a complex comprising a nucleic acid (e.g., an RNP complex), refers to the translocation of the nucleic acid sequence, the polypeptide or the complex from outside a cell to inside the cell. In some cases, introducing refers to translocation of the nucleic acid or the complex from outside the cell to inside the nucleus of the cell.

Any of the methods described herein can be used to deliver an agent to a cell in vitro, ex vivo or in vivo. See, for example, Skipper (2019), Toward In Vivo Gene Therapy Using CRISPR, Methods Mol. Biol. 1961:293-306, and Gillmore et al. (2021) CRISPR-Cas9 In Vivo Gene Editing for Transthyretin Amyloidosis, N. Engl. J. Med. 385:493-502, for in vivo applications.

In any of the methods provided herein, the cell can be a prokaryotic or a eukaryotic cell. In some embodiments, the cell is a mammalian cell, for example, a human cell. One or more LNPs comprising an RNA-guided endonuclease mRNA and a gRNA can be administered to a subject in need thereof. One or more LNPs comprising an RNP complex can be administered to the subject, wherein the RNP comprises an RNA-guided endonuclease and a gRNA, and, optionally, a nucleic acid template. In some embodiments, the LNP is targeted to one or more target cells or tissues in the subject. For example, the target tissue can be, but is not limited to, liver, eye, endothelial tissue, lung, blood, muscle or kidney or a specific cell type in the liver, eye, endothelial tissue, lung, blood, muscle or kidney.

Ex vivo methods include, but are not limited to, obtaining one or more cells from a subject having a disease or disorder, editing one or more target nucleotide sequences in the one or more cells ex vivo, using an LNP comprising (1) an RNA-guided endonuclease mRNA, and a gRNA or (2) an LNP comprising an RNP complex comprising an RNA-guided endonuclease and a gRNA (optionally, with a nucleic acid template), and re-introducing the one or more cells with the edited target nucleotide sequence back into the subject having the disease or disorder. In any of the ex vivo methods described herein, the cells can be cultured, and/or expanded before or after editing.

The LNP compositions disclosed herein can be administered to a subject in a number of ways depending on whether local or systemic treatment is desired, and on the cell/tissue/area to be treated. The compositions are administered via any of several routes of administration, including orally, intranasally, via inhalation, via nebulizer, parenterally, intravenously, intraperitoneally, intracranially, intraspinally, intrathecally, intraventricularly, intramuscularly, subcutaneously, intracavity or transdermally. Pharmaceutical compositions can also be delivered locally to the area in need of treatment, for example by topical application or local injection. The pharmaceutical compositions can also be delivered via pump or at a surgical site. Effective doses for any of the administration methods described herein can be extrapolated from dose-response curves derived from in vitro or animal model test systems.

The amount of LNPs or the amount of active agent in the LNPs administered depends upon a number of factor including the disease state of the subject being treated or prevented and the judgment of the clinician but is generally between about 0.01 and about 150 mg of the agent per kilogram of body weight, preferably between about 0.1 and about 20 mg/kg of body weight, about 0.1 to about 10 mg/kg of body weight or about 0.1 to about 5 mg/kg of body weight, which may be administered in a single dose or in the form of individual doses, such as from 1 to 4 times per day. Administration can be performed for 1, 2, 3, 4, 5, 6, 7, 8, 9, 10, 11, 12, 13, 14, 15, 16, 17, 18, 19, 20 or more days. One of skill in the art can adjust the dosage based on specific characteristics of the agent and the subject receiving it.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

The following examples are provided by way of illustration only and not by way of limitation. Those of skill in the art will readily recognize a variety of non-critical parameters that could be changed or modified to yield essentially the same or similar results.

### Ionizable Lipid Formulations

PCTA-1 (Formula II) was used in a formulation comprising 50% PCTA-1, 10% Distearoylphosphatidylcholine (DSPC), 38% cholesterol, and 2% pegylated myristoyl diglyceride (DMG-PEG2000). This formulation can be adjusted to include about 20% to about 80% PCTA-1 depending on the application, with a helper lipid percentage between about 5 to about 20%, a cholesterol percentage between 10%-60%, and polyethylene glycol (PEG) between about 0.5% to about 5%.

For applications involving nucleic acid delivery, the nucleic acid (for example, mRNA or a combination of mRNA and gRNA in the case of gene editing) was incorporated at an N:P (nitrogen to phosphate) ratio of 5.5 such that there were 5.5 times as many ionizable amines (from PCTA-1, as the amine of DSPC is not ionizable) as there are phosphate groups in the nucleic acid. The LNPs were generated by combining a solution of lipids in ethanol with a solution of nucleic acid in buffer, using a microfluidic mixer to rapidly mix the solutions. The buffer must have an acidic pH but can otherwise vary, with the most effective buffer observed to be 25 mM citrate buffer at pH 4.5. The concentration of the lipids in ethanol can also vary depending on desired attributes but is generally in the range of 1-50 mg/mL lipid. The concentration of nucleic acid is dictated by the N:P ratio, the concentration of the lipid in the buffer, and the ratio of ethanol to buffer. The ratio of ethanol to buffer is also variable, with ratios of 3:1 buffer:ethanol to 1:1 being effective. Microfluidic mixing can be done at a variety of flow rates, with a flow rate of 1-10 mL/minute being most effective. A number of mixing architectures are also available, for example, a staggered herringbone mixing architecture. In this example, a Dolomite micromixer chip was used for PCTA-1.

PCTA-1 was used in LNP formulations to demonstrate effective delivery of cas9 mRNA and guide RNA to the liver in vivo. High editing efficiency was achieved for two different genes. Mice were dosed via tail vein, and on day five, livers were harvested. Percent editing was determined by isolating gDNA from homogenized whole livers, and next-generation targeted amplicon sequencing was used to quantify editing events.

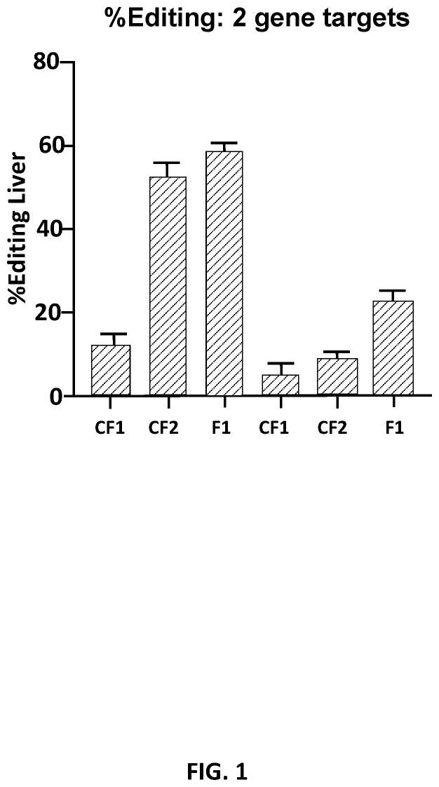

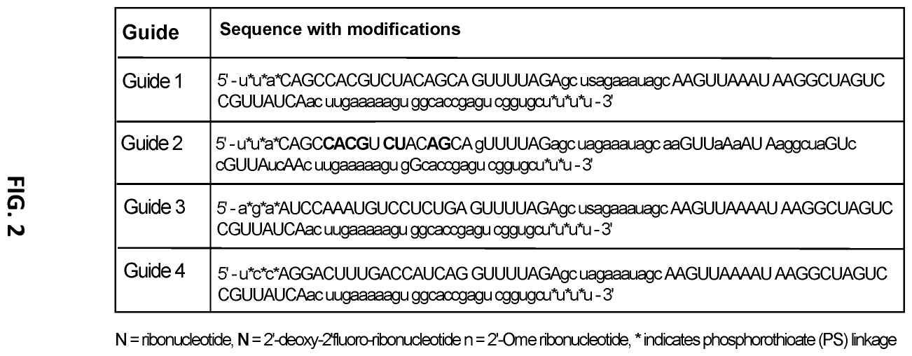

As shown in FIG. 1, Formulation 1 (F1) (a formulation containing PCTA-1 (Formula II)) was compared to Comparative Formulation 1 (CF1) and Comparative formulation 2 (CF2) generated using Formula III for CF1 (See, for example, Finn et al. Cell Reports 22, 2227-2235 (2018)) and Formula IV (heptadecan-9-yl 8-((2-hydroxyethyl)(8-(nonyloxy)-8-oxooctyl)amino) octanoate; See, for example, Sabnis et al. Molecular Therapy 26(6): 1509-1519 (2018)) for CF2 (both formulated according to literature reports).

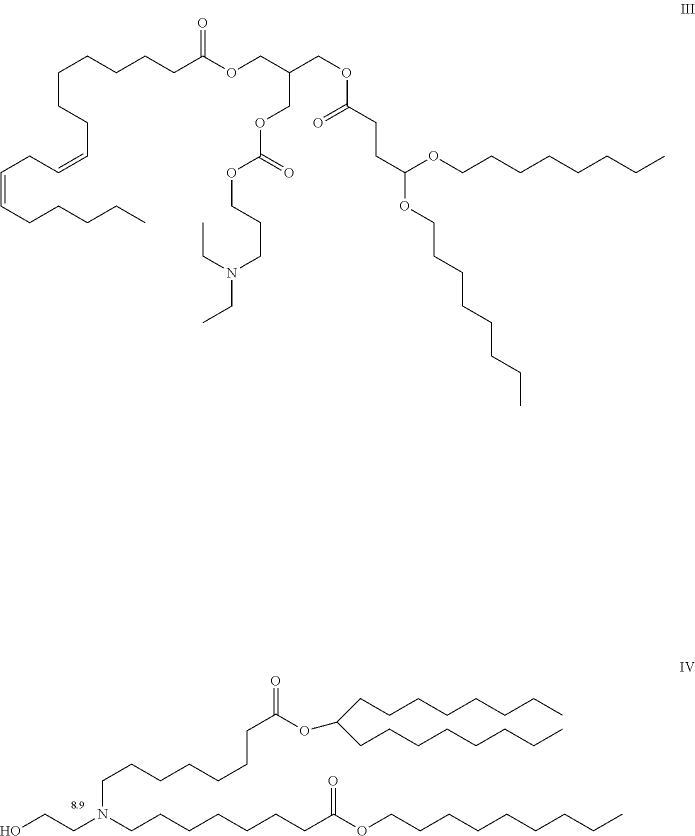 [SMILES: CCCCC/C=C\C/C=C\CCCCCCCC(=O)OCC(COC(=O)CCC(OCCCCCCCC)OCCCCCCCC)COC(=O)OCCCN(CC)CC ; CCCCCCCCCOC(=O)CCCCCCCN(CCO)CCCCCCCC(=O)OC(CCCCCCCC)CCCCCCCC]

All formulations packaged Cas9 mRNA and an sgRNA targeting either TTR (first three columns from the left) or PCSK9 (fourth through sixth columns from the left). All formulations were dosed at 0.7 mg/kg in groups of three mice, with organ collection after five days. When targeting TTR, F1 achieved the highest editing, showing improvement over CF2 and an approximate 5× improvement over CF1. When targeting liver PCSK9, F1 achieved significantly higher editing (2-3×), as compared to CF2 and CF1, with the same sgRNA.

### Modified Guide RNAs

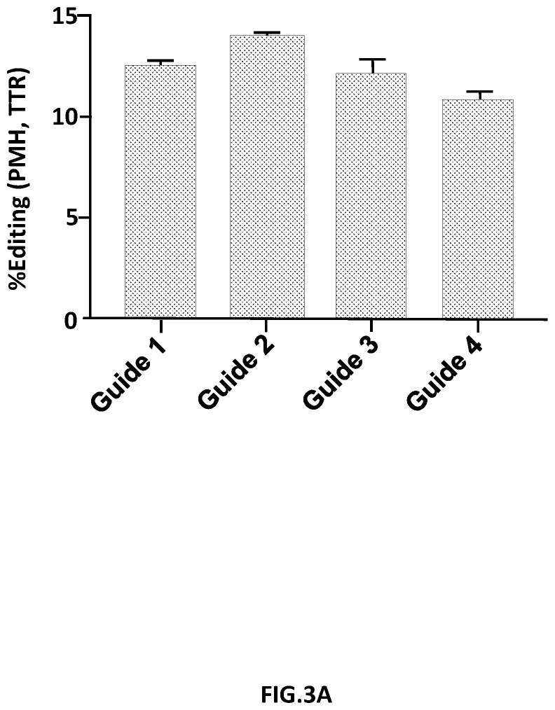

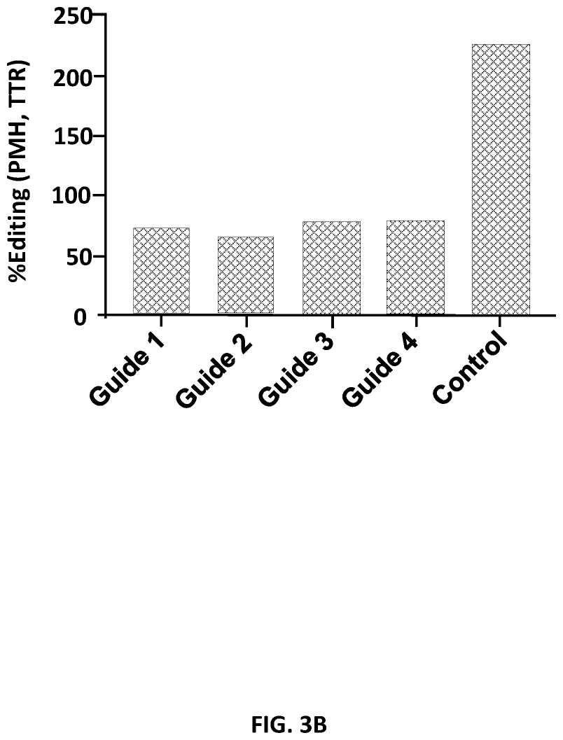

Numerous chemical modification patterns were designed and tested for gene editing in cells and in in vivo mouse models. The design modified 69 of the 100 nucleotides found within the sgRNA (FIG. 2). Sugar modifications of 2′ Ome and 2′F along with thioester modifications to the phosphate backbone were used to increase nuclease resistance in vitro and improve in vivo delivery of active sgRNAs. Three sgRNAs were designed and compared to previously published modifications (Guide 1). Testing of sgRNAs in vitro, using primary mouse hepatocyte cells, showed similar percent editing and reductions in cell supernatant target protein levels. Briefly, Cas9 mRNA and sgRNA were complexed using MessengerMax transfection reagent and added to seeded mouse hepatocyte cells, then incubated for 48 hrs before isolating genomic DNA for sequencing. Genomic DNA was used as the input for amplifying the target area of interest around the expected gene editing, and barcoded using unique adaptor sequences to allow combining samples for next-generation sequencing. FIG. 3A shows the percent editing for the target of interest. All guides showed similar editing in vitro.

Target protein levels in cell culture supernatant were measured with a commercially available ELISA kit, using the manufacturer's instructions (FIG. 3B). Results show similar decreases for all sgRNAs compared to control.

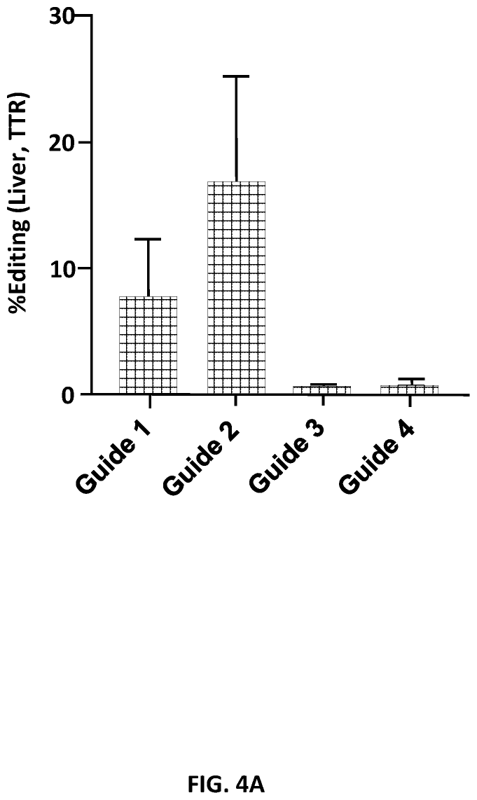

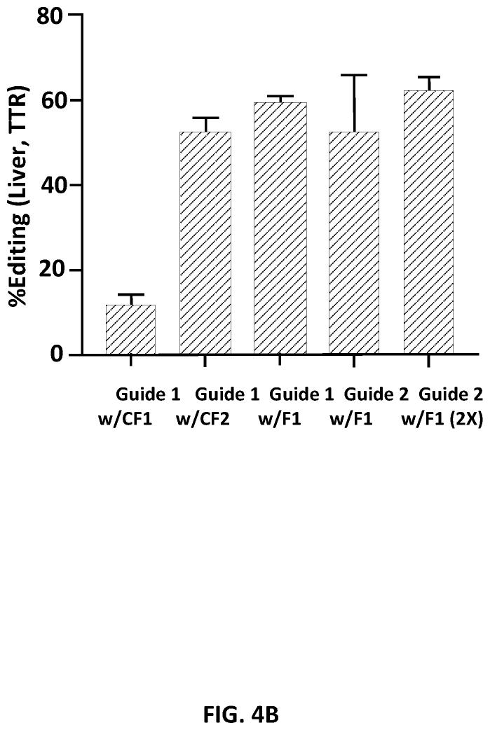

All sgRNAs were screened initially in vivo using a CD1 mouse model. Briefly, mice were injected via tail vein, and on day 5, plasma, liver and spleens were harvested. Percent editing was determined from genomic DNA isolated from homogenized whole liver or spleens. Plasma was used for protein determinations by ELISA. Results showed that Guide 2, with additionally designed chemical modifications, showed a 2-fold increase in percent editing using a nanoparticle formulation (FIG. 4A) described in the literature. A subsequent in vivo study using a nanoparticle formulation described herein showed that ˜60% liver editing, was achieved (FIG. 4B). This is likely near saturation, as hepatocytes constitute approximately 70% of liver cells.

What is claimed is:

1. A lipid nanoparticle (LNP) comprising an ionizable lipid of Formula I 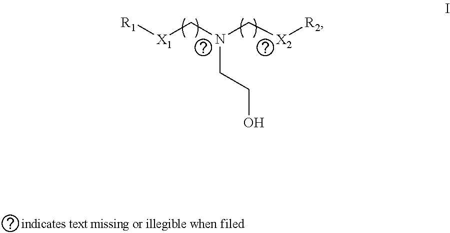
wherein, R₁ and R₂ are independently selected from the group consisting of 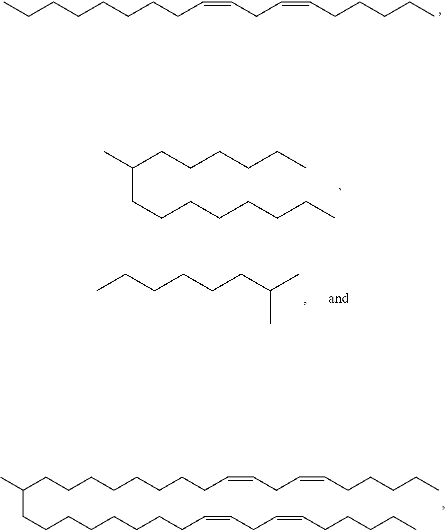
wherein X₁ and X₂ are hydrolyzable linkers, wherein (a) is 2-9, and wherein (b) is 2-9.

2. The LNP of claim 1, wherein the hydrolyzable linker is an ester.

3. The LNP of claim 1, wherein the ionizable is a lipid of Formula II

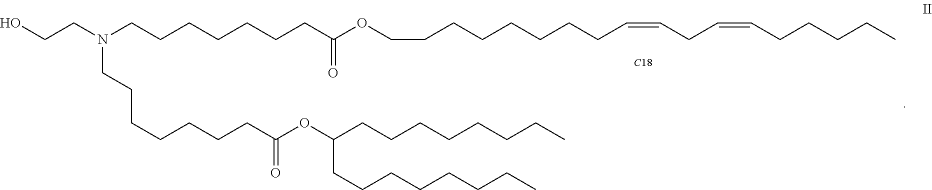

4. The LNP of claim 1, further comprising cholesterol, a helper phospholipid and a polyethylene glycol (PEG)-lipid.

5. The LNP of claim 1, wherein about 20% to about 80% of the total lipid content of the LNP is the ionizable lipid of Formula I.

6. The LNP of claim 1, wherein the LNP comprises:
(a) 20%-80% ionizable lipid of Formula I,
(b) 5%-20% helper lipid,
(c) 10%-60% cholesterol, and
(d) 0.5%-5% PEG.

7. The LNP of claim 1, further comprising a polypeptide, a nucleic acid, or a combination thereof.

8. The LNP of claim 7, wherein a nucleic acid encoding a heterologous polypeptide is encapsulated in the LNP.

9. The LNP of claim 8, wherein the nucleic acid is an mRNA.

10. The LNP of claim 9, wherein the mRNA encodes an RNA-guided endonuclease.

11. The LNP of claim 10, wherein the RNA-guided endonuclease is a Cas9 nuclease.

12. The LNP of claim 9, wherein the LNP further comprises a guide RNA.

13. The LNP of claim 12, wherein the guide RNA is a modified guide RNA.

14. The LNP of claim 13, wherein the guide RNA comprises one or more modifications selected from the group consisting of a backbone modification, a sugar modification, a base modification, and any combination thereof.

15. The LNP of claim 14, wherein the sugar modification is a 2′ OMe or a 2′F modification of the 2′ ribose group.

16. The LNP of claim 14, wherein the backbone modification is a thioester modification.

17. The LNP of claim 13, wherein the guide RNA comprises a nucleic acid sequence of 100 nucleotides, wherein 69 of the 100 nucleotides are modified.

18. The LNP of claim 10, wherein the mRNA encoding the RNA-guided endonuclease and the guide RNA are encapsulated separately and then combined to form one or more LNPs comprising the RNA-guided endonuclease and the guide RNA.

19. The LNP of claim 18, wherein the mRNA encoding the RNA-guided endonuclease and the guide RNA are co-encapsulated in the one or more LNPs.

20. The LNP of claim 19, wherein the ratio of nitrogen groups in the ionizable lipid to phosphate groups in the mRNA and/or gRNA in the LNP is from about 4 to about 6.

21. The LNP of claim 10, wherein the LNP further comprises at least one template sequence.

22. A pharmaceutical composition comprising the LNP of claim 1.

23. A method of delivering a heterologous polypeptide to a cell comprising contacting a cell with the LNP of claim 8 or a pharmaceutical composition comprising the LNP of claim 8.

24. A method of editing the genome of a cell comprising contacting a cell with the LNP of claim 10 or a pharmaceutical composition comprising the LNP of claim 10.

25. (canceled)

26. (canceled)

27. A genetically modified cell made by the method of claim 26.


SEQUENCE LISTING (3 sequences)

SEQ ID NO: 1 - skipped

SEQ ID NO: 2 - RNA, 100 nt, synthetic construct
  source 1..100: mol_type = other RNA
agaatccaaatgtcctctgagttttagagctagaaatagcaagttaaaataaggctagtccgttatcaacttgaaaaagtggcaccgagtcggtgctttt

SEQ ID NO: 3 - RNA, 100 nt, Synthetic construct
  source 1..100: mol_type = other RNA
tccaggactttgaccatcaggttttagagctagaaatagcaagttaaaataaggctagtccgttatcaacttgaaaaagtggcaccgagtcggtgctttt